(19) United States
(12) Patent Application Publication
(10) Pub. No.: US 2012/0108780 A1
(43) Pub. Date: May 3, 2012

(54) METHOD FOR PRODUCING POLYOLS ON THE BASIS OF RENEWABLE RESOURCES

(75) Inventors: Andreas Kunst, Ludwigshafen (DE); Michael Schelper, Ludwigshafen (DE); Joaquim Henrique Teles, Waldsee (DE); Berend Eling, Lemfoerde (DE); Jenny Reuber, Mannheim (DE); Gerd-Dieter Tebben, Mannheim (DE)

(73) Assignee: BASF SE, Ludwigshafen (DE)

(21) Appl. No.: 13/382,991

(22) PCT Filed: Jul. 9, 2010
(86) PCT No.: PCT/EP10/59883

§ 371 Date: Jan. 9, 2012

(30) Foreign Application Priority Data
Jul. 10, 2009 (EP) 09165148.9

(51) Int. Cl. C08G 18/28; C07C 43/11; C07C 41/03

(52) U.S. Cl. 528/85; 568/620; 568/623

(57) ABSTRACT

A method for producing a polyol, the method including:

(a) reacting at least one selected from the group consisting of an unsaturated natural fat, an unsaturated natural fatty acid, and a fatty acid ester with dinitrogen monoxide, to obtain a first intermediate;

(b) reacting the first intermediate with a hydrogenation reagent, to obtain a second intermediate;

(c) reacting the second intermediate with at least one alkylene oxide, to obtain a polyol.

The invention relates to a method for producing polyols based on natural oils, in particular for producing polyurethanes.

Polyurethanes are used in many technical fields. They are usually produced by reacting polyisocyanates with compounds having at least two hydrogen atoms that are reactive with isocyanate groups, in the presence of blowing agents, and optionally catalysts and customary auxiliaries and/or additives.

More recently, polyurethane starting components based on renewable raw materials have been gaining importance. Particularly in the case of the compounds having at least two hydrogen atoms that are reactive with isocyanate groups, it is possible to use natural oils and fats, which are usually chemically modified prior to use in polyurethane applications, in order to introduce at least two hydrogen atoms that are reactive with isocyanate groups. During the chemical modifications, in most cases natural fats and/or oils are hydroxy-functionalized and optionally modified in one or more further steps. Examples of applications of hydroxy-functionalized fat and/or oil derivatives in PU systems which may be mentioned are, for example, WO 2006/116456 and WO 2007/130524.

The reactive hydrogen atoms necessary for use in the polyurethane industry have to be introduced into most of the naturally occurring oils as described above by means of chemical methods. For this purpose, according to the prior art, there are essentially methods which utilize the double bonds that occur in the fatty acid esters of numerous oils. Firstly, fats can be oxidized by reaction with percarboxylic acids in the presence of a catalyst to give the corresponding fatty acid or fatty acid epoxides. The subsequent acid- or base-catalyzed ring-opening of the oxirane rings in the presence of alcohols, water, carboxylic acids, halogens or hydrohalides leads to the formation of hydroxy-functionalized fats or fat derivatives (WO 2007/127379 and US 2008076901). The disadvantage of this method is that very corrosion-resistant materials have to be used for the first reaction step (epoxidation) since said step is carried out on an industrial scale with corrosive performic acid or with peracetic acid. Moreover, the dilute percarboxylic acid which is produced has to be concentrated again by distillation and returned after the production for an economic method, which necessitates the use of corrosion-resistant and thus energy- and cost-intensive distillation apparatuses.

A further hydroxy functionalization option is to firstly hydroformylate the unsaturated fat or fatty acid derivative in the first reaction step in the presence of a cobalt- or rhodium-containing catalyst with a mixture of carbon monoxide and hydrogen (synthesis gas), and then to hydrogenate the aldehyde functions inserted by this reaction step with a suitable catalyst (e.g. Raney nickel) to give hydroxy groups (cf. WO 2006/12344 A1 or also J. Mol. Cat. A, 2002, 184, 65 and J. Polym. Environm. 2002, 10, 49). With this reaction route, however, it has to be taken into consideration that the use of a catalyst and of a solvent is necessary at least also for the first reaction step of the hydroformylation, and these likewise have to be recovered again and purified or regenerated for an economic production.

EPI 170274A1 describes a method for producing hydroxy oils by oxidizing unsaturated oils in the presence of atmospheric oxygen. It is a disadvantage that, using this method, it is not possible to achieve high degrees of functionalization and that the reactions have to take place at high temperatures, which leads to the partial decomposition of the fat structure.

A further option for introducing hydroxy functions into fats is to cleave fat or the fat derivative in the presence of ozone, and then to reduce to the hydroxy fat derivative (cf. Biomacromolecules 2005, 6, 713; J. Am. Oil Chem. Soc. 2005, 82, 653 and J. Am.

Oil Chem. Soc. 2007, 84, 173). This process too has to take place in a solvent and is usually carried out at low temperatures (−10 to 0° C.), which likewise results in comparatively high production costs. The safety-related characteristics of this process moreover require the cost-intensive provision of safety measures, such as measurement and control technology or compartmentation.

In Adv. Synth. Catal. 2007, 349, 1604, the ketonization of fats by means of nitrous oxide is described. The ketone groups can be converted into hydroxyl groups. However, there is no indication at all of the further processing of these products.

One option for producing polyols based on renewable raw materials for polyurethanes consists in reacting unsaturated naturally occurring fats such as, e.g. soyabean oil, sunflower oil, rapeseed oil, etc. or corresponding fat derivatives such as fatty acids or monoesters thereof by corresponding derivatization to give hydroxy-functionalized fats or fatty acid derivatives. These materials can either be used directly for the appropriate

PU application or alternatively following the additional addition reaction of alkylene oxides onto the OH functions in the hydroxy-functionalized fat or fat derivative. Examples of the reaction of hydroxy fat derivatives with alkylene oxides and the use of the reaction products in polyurethane applications can be found, for example, in WO 2007/143135 and EP1537159. The addition reaction takes place here in most cases with the help of so-called double-metal cyanide catalysts.

It was the object of the present invention to provide polyols based on renewable raw materials, in particular based on natural fats and fatty acid derivatives, for polyurethane applications which are available in a cost-effective manner and in which, as a result of very simple, adaptation of the reaction parameters, highly diverse functionalities can be covered and the products are thus available for a broad area of application. In particular, the production of the oils and fats should be possible by a simple method without using costly raw materials (catalysts and solvents).

The object was achieved by oxidizing unsaturated natural fats such as soyabean oil, sunflower oil, rapeseed oil, or corresponding fatty acid derivatives, in a first step in the presence of dinitrogen monoxide, also termed nitrous oxide, to give ketonized fats or fatty acid derivatives, and reducing these in a further reaction step in the presence of hydrogenation reagents and optionally in the presence of a suitable catalyst to give hydroxy fats. The hydroxyl groups are reacted in a further step with alkylene oxides.

Accordingly, the invention provides a method for producing polyols based on renewable raw materials, comprising the steps
a) reacting unsaturated natural fats, unsaturated natural fatty acids and/or fatty acid esters with dinitrogen monoxide,
b) reacting the product obtained in step a) with a hydrogenation reagent
c) reacting the reaction product from step b) with alkylene oxides.

These materials can be used directly as polyol component in highly diverse applications, e.g. in the corresponding PU application.

Preferably, the natural, unsaturated fats are selected from the group comprising castor oil, grapeseed oil, black caraway oil, pumpkin seed oil, borage seed oil, soya oil, wheat germ oil, rapeseed oil, sunflower oil, peanut oil, apricot kernel oil, pistachio kernel oil, almond oil, olive oil, macadamia nut oil, avocado oil, sea buckthorn oil, sesame oil, hemp oil, hazelnut oil, evening primrose oil, wild rose oil, safflower oil, walnut oil, palm oil, fish oil, coconut oil, tall oil, corn germ oil, linseed oil.

Preferably, the fatty acids and fatty acid esters are selected from the group comprising myristoleic acid, palmitoleic acid, oleic acid, vaccenic acid, petroselinic acid, gadoleic acid, erucic acid, nervonic acid, linoleic acid, α- and γ-linolenic acid, stearidonic acid, arachidonic acid, timnodonic acid, clupanodonic acid and cervonic acid, and esters thereof.

As fatty acid esters it is possible to use either fully or partially esterified mono- or polyhydric alcohols. Suitable mono- or polyhydric alcohols are methanol, ethanol, propanol, isopropanol, butanol, ethylene glycol, propylene glycol, diethylene glycol, dipropylene glycol, glycerol, trimethylolpropane, pentaerythritol, sorbitol, sucrose and mannose.

Particularly preferably, the natural, unsaturated fats are selected from the group comprising castor oil, soya oil, palm oil, sunflower oil and rapeseed oil. In particular, soya oil, palm oil, sunflower oil and rapeseed oil are used. These compounds are used on an industrial scale in particular also for the production of biodiesel.

Besides the specified oils, it is also possible to use those oils which have been obtained from genetically modified plants and have a different fatty acid composition. Besides the specified oils, as described above, the corresponding fatty acids or fatty acid esters can likewise be used.

The reaction steps a) to c) can be carried out independently of one another and optionally also at different times and in different places. However, it is possible to carry out three method steps directly one after the other. In this connection, it is also possible to carry out the method in an entirely continuous manner.

Step a) is preferably carried out under pressure, in particular in a pressure range from 10-300 bar and elevated temperature, in particular in a temperature range from 200 to 350° C. Here, the oil or fat can be used without dilution or in solutions of suitable solvents, such as cyclohexane, acetone or methanol. The reaction can take place in a stirred reactor of any design or a tubular reactor; a reaction in any other desired reactor system is possible in principle. The nitrous oxide used can be used as pure substance or as a mixture with gases that are inert under the reaction conditions, such as nitrogen, helium, argon or carbon dioxide. Here, the amount of inert gases is at most 50% by volume.

When the reaction is complete, the reaction mixture is cooled for the further processing, if necessary the solvent is removed, for example by means of distillation or extraction, and passed to step b) with or without further work-up.

The reaction product from step a) is hydrogenated in step b). This too takes place by customary and known methods. For this, the preferably purified organic phase from step a) is reacted, preferably in the presence of a suitable solvent, with a hydrogenation reagent. If hydrogen is used as hydrogenation reagent, the presence of a catalyst is required. For this, the organic phase is then reacted at a pressure of from 50 to 300 bar, in particular at 90 to 150 bar, and a temperature of from 50 to 250° C., in particular 50 to 120° C., in the presence of hydrogenation catalysts. Hydrogenation catalysts which can be used are homogeneous or preferably heterogeneous catalysts. Preferably, catalysts comprising ruthenium are used. Moreover, the catalysts can consist of other metals, for example of metals of group 6-11, such as, e.g. nickel, cobalt, copper, molybdenum, palladium or platinum. The catalysts can be water-moist. The hydrogenation is preferably carried out in a fixed bed.

Besides the use of hydrogen as hydrogenation reagent in step b), it is also possible to use, for example, complex hydrides such as e.g. lithium aluminum hydride, sodium or lithium borohydride. This is described, for example, in Organikum—Organisch-chemisches Grundpraktikum [Organic Chemistry—organic chemistry basic practice], VEB Deutscher Verlag der Wissenschaften, Berlin 1967, 6th edition, pp. 481-484. In this case, the presence of an anhydrous solvent is required. Suitable solvents are all customary solvents which do not react with the hydrogenation reagent. For example, alcohols such as methanol, ethanol, n-propanol, isopropanol or butanol can be used. Further solvents are linear or cyclic ethers, such as tetrahydrofuran or diethyl ether.

After the hydrogenation, the organic solvents, if used the catalyst and if required water, are separated off. If required, the product is purified.

The product obtained in this way is reacted in a further process step c) with alkylene oxides.

The reaction with the alkylene oxides usually takes place in the presence of catalysts. In this regard, in principle all alkoxylation catalysts can be used, for example alkali metal hydroxides or Lewis acids. However, multi-metal cyanide compounds, so-called DMC catalysts, are preferably used.

The DMC catalysts used are generally known and described, for example, in EP 654 302, EP 862 947 and WO 00/74844.

The reaction with alkylene oxides is usually carried out with a DMC concentration of 10-1000 ppm, based on the end product. The reaction is particularly preferably carried out with a DMC concentration of 20-200 ppm. The reaction is very particularly preferably carried out with a DMC concentration of 50-150 ppm.

The addition reaction of the alkylene oxides takes place under the customary conditions, at temperatures in the range from 60 to 180° C., preferably between 90 and 140° C., in particular between 100 and 130° C. and pressures in the range from 0 to 20 bar, preferably in the range from 0 to 10 bar and in particular in the range from 0 to 5 bar. The mixture of starting substance and DMC catalyst can be pretreated by stripping prior to the start of the alkoxylation in accordance with the teaching of WO 98152689.

Prior to the addition reaction of the alkylene oxides, the products from step b) are in most cases subjected to a drying. This takes place in most cases by stripping, for example using inert gases, such as nitrogen or steam, as stripping gases.

Alkylene oxides which can be used are all known alkylene oxides, for example ethylene oxide, propylene oxide, butylene oxide, styrene oxide. In particular, the alkylene oxides used are ethylene oxide, propylene oxide and mixtures of said compounds.

In one embodiment of the invention, the specified alkylene oxides are used in the mixture with monomers which are not alkylene oxides. Examples thereof are cyclic anhydrides, lactones, cyclic esters, carbon dioxide or oxetanes. In the case of the use of lactones as comonomers, the reaction temperature during the addition reaction of the alkylene oxides should be >150° C.

The oxidized and hydrogenated natural fats or fat derivatives from method step b) can preferably be reacted on their own with the alkylene oxides.

However, it is also possible to carry out the reaction with the alkylene oxides in the presence of so-called co-starters. Co-starters which can be used are preferably alcohols, such as higher-functional alcohols, in particular sugar alcohols, for example sorbitol, hexitol and sucrose, but in most cases di- and/or trifunctional alcohols or water, either as individual substance or as a mixture of at least 2 of the specified co-starters. Examples of difunctional starter substances are ethylene glycol, diethylene glycol, propylene glycol, dipropylene glycol, butanediol-1,4 and pentanediol-1,5. Examples of trifunctional starter substances are trimethylolpropane, pentaerythritol and in particular glycerol. The starter substances can also be used in the form of alkoxylates, in particular those with a molecular weight Mn in the range from 62 to 15 000 g/mol. In principle, the use of castor oil or of alkoxylated castor oil is also possible here.

The addition reaction of the alkylene oxides during the production of the polyether alcohols used for the method according to the invention can take place by known methods. Thus, it is possible that only one alkylene oxide is used for producing the polyether alcohols. When using a plurality of alkylene oxides, a so-called blockwise addition reaction is possible, in which the alkylene oxides are added individually one after the other, or a so-called random addition, also termed heteric, in which the alkylene oxides are added together. It is also possible, during the production of the polyether alcohols, to incorporate both blockwise and also random sections into the polyether chain. Furthermore, gradient-like or alternating addition reactions are possible, as has been described, for example, in DE 19960148.

In one embodiment of the invention, the starters are passed to the reaction continuously during the reaction. This embodiment is described, for example, in WO 98/03571. It is also possible to continuously meter in the optionally co-used co-starters. It is also possible to carry out the entire reaction with the alkylene oxides continuously, as likewise described in WO 98/03571.

In a further embodiment of the invention, the alkoxylation can also be carried out as a so-called heel process. This means that the reaction product is introduced as initial charge again as starting material in the reactor.

When the addition reaction of the alkylene oxides is complete, the polyether alcohol is worked up by customary methods by removing the unreacted alkylene oxides and readily volatile constituents, usually by distillation, steam or gas stripping and/or other methods of deodorization. If necessary, a filtration can also take place.

The polyether alcohols according to the invention from process step c) preferably have an average functionality of from 2 to 6, in particular from 2 to 4, and a hydroxyl number in the range between 20 and 120 mg KOH/g. Consequently, they are suitable in particular for flexible PU foam and also for PU adhesives, sealants and elastomers.

Depending on the type of fat or fat derivative used in process step a), the polyether alcohols according to the invention from process step b) have an average functionality of 2 to 6, in particular from 2 to 4, and a hydroxyl number in the range between 50 and 300 mg KOH/g. The structures are suitable in particular for producing polyurethanes, in particular for flexible polyurethane foams, rigid polyurethane foams and polyurethane coatings. During the production of rigid polyurethane foams and polyurethane coatings, it is in principle also possible to use those polyols onto which no alkylene oxides have been added, i.e. polyols based on renewable raw materials, for the production of which only method steps a) and b) have been carried out. In the case of the production of flexible polyurethane foams, compounds of this type lead, on account of their low chain lengths, to undesired crosslinking and are therefore less suitable.

The polyurethanes are produced by reacting the polyether alcohols produced by the method according to the invention with polyisocyanates.

The polyurethanes according to the invention are produced by reacting polyisocyanates with compounds having at least two hydrogen atoms that are reactive with isocyanate groups. In the case of the production of foams, the reaction takes place in the presence of blowing agents.

The following details relate to the starting compounds used.

Suitable polyisocyanates are the aliphatic, cycloaliphatic, araliphatic and preferably aromatic polyvalent isocyanates known per se.

Specifically, mention may be made by way of example to: alkylene diisocyanates having 4 to 12 carbon atoms in the alkylene radical, such as e.g. hexamethylene diisocyanate-1,6; cycloaliphatic diisocyanates, such as e.g. cyclohexane 1,3- and 1,4-diisocyanate, and any desired mixtures of these isomers, 2,4- and 2,6-hexahydrotoluene diisocyanate, and the corresponding isomer mixtures, 4,4′-, 2,2′- and 2,4′-dicyclohexylmethane diisocyanate, and also the corresponding isomer mixtures, araliphatic diisocyanates, such as e.g. 1,4-xylylene diisocyanate and xylylene diisocyanate isomer mixtures, but preferably aromatic di- and polyisocyanates, such as e.g. 2,4- and 2,6-toluene diisocyanate (TDI) and the corresponding isomer mixtures, 4,4′-, 2,4′- and 2,2′-diphenylmethane diisocyanate (MDI) and the corresponding isomer mixtures, mixtures of 4,4′- and 2,4′-diphenylmethane diisocyanates, polyphenyl-polymethylene polyisocyanates, mixtures of 4,4′-, 2,4′- and 2,2′-diphenylmethane diisocyanates and polyphenyl-polymethylene polyisocyanates (crude MDI) and mixtures of crude MDI and toluylene diisocyanates. The organic di- and polyisocyanates can be used individually or in the form of mixtures.

So-called modified polyvalent isocyanates, i.e. products which are obtained by chemical reaction of organic di- and/or polyisocyanates, are also often used. By way of example, mention may be made of di- and/or polyisocyanates comprising isocyanurate and/or urethane groups. Specifically of suitability are, for example, urethane-group-comprising organic, preferably aromatic, polyisocyanates with NCO contents of from 33 to 15% by weight, preferably from 31 to 21% by weight, based on the total weight of the polyisocyanate.

The polyols produced by the method according to the invention can be used in combination with other compounds having at least two hydrogen atoms that are reactive with isocyanate groups.

As compounds having at least two hydrogen atoms that are reactive with isocyanate and which can be used together with the polyols produced by the method according to the invention, use is made in particular of polyether alcohols and/or polyester alcohols.

In the case of the production of rigid polyurethane foams, in most cases at least one polyether alcohol is used which has a functionality of at least 4 and a hydroxyl number greater than 250 mg KOH/g.

The polyester alcohols used together with the polyols produced by the method according to the invention are in most cases produced by condensation of polyfunctional alcohols, preferably diols, having 2 to 12 carbon atoms, preferably 2 to 6 carbon atoms, with polyfunctional carboxylic acids having 2 to 12 carbon atoms, for example succinic acid, glutaric acid, adipic acid, suberic acid, azelaic acid, sebacic acid, decanedicarboxylic acid, maleic acid, fumaric acid and preferably phthalic acid, isophthalic acid, terephthalic acid and the isomeric naphthalenedicarboxylic acids.

The polyether alcohols used together with the polyols produced by the method according to the invention have in most cases a functionality between 2 and 8, in particular 4 to 8.

The polyhydroxyl compounds used are in particular polyether polyols which are produced by known methods, for example by anionic polymerization of alkylene oxides in the presence of alkali metal hydroxides.

The alkylene oxides used are preferably ethylene oxide and 1,2-propylene oxide. The alkylene oxides can be used individually, alternately one after the other or as mixtures.

Suitable starter molecules are, for example: water, organic dicarboxylic acids, such as e.g. succinic acid, adipic acid, phthalic acid and terephthalic acid, aliphatic and aromatic, optionally N-mono-, N,N- and N,N′-dialkyl-substituted diamines having 1 to 4 carbon atoms in the alkyl radical, such as e.g. optionally mono- and dialkyl-substituted ethylenediamine, diethylenetriamine, triethylenetetramine, 1,3-propylenediamine, 1,3- or 1,4-butylenediamine, 1,2-, 1,3-, 1,4-, 1,5- and 1,6-hexamethylenediamine, aniline, phenylenediamines, 2,3-, 2,4-, 3,4- and 2,6-toluenediamine and 4,4′-, 2,4′- and 2,2′-diaminodiphenylmethane.

Also suitable as starter molecules are: alkanolamines, such as e.g. ethanolamine, N-methyl- and N-ethylethanolamine, dialkanolamines, such as e.g. diethanolamine, N-methyl- and N-ethyldiethanolamine and trialkanolamines such as e.g. triethanolamine and ammonia.

Polyhydric, in particular di- and/or trihydric alcohols, such as ethanediol, propanediol-1,2 and -1,3, diethylene glycol, dipropylene glycol, butanediol-1,4, hexanediol-1,6, glycerol, pentaerythritol, sorbitol and sucrose, polyhydric phenols, such as e.g. 4,4′-dihydroxydiphenylmethane and 4,4′-dihydroxydiphenylpropane-2,2, resols, such as e.g. oligomeric condensation products of phenol and formaldehyde and Mannich condensates of phenols, formaldehyde and dialkanolamines, and melamine.

The polyetherpolyols have a functionality of preferably 3 to 8 and in particular 3 and 6 and hydroxyl numbers of preferably 120 mg KOH/g to 770 mg KOH/g and in particular 240 mg KOH/g to 570 mg KOH/g.

The compounds having at least two hydrogen atoms that are reactive with isocyanate groups also include the optionally co-used chain extenders and crosslinkers. To modify the mechanical properties, however, the addition of difunctional chain extending agents, tri- and higher-functional crosslinking agents or optionally also mixtures thereof can prove to be advantageous. Alkanolamines and in particular diols and/or triols with molecular weights less than 400, preferably 60 to 300, are preferably used as chain extending agents and/or crosslinking agents.

If chain extending agents, crosslinking agents or mixtures thereof are used for producing the polyurethanes, these are expediently used in an amount of from 0 to 20% by weight, preferably 2 to 5% by weight, based on the weight of the compounds having at least two hydrogen atoms that are reactive with isocyanate groups.

As blowing agent, preference is given to using water, which reacts with isocyanate groups with the elimination of carbon dioxide. Instead of, but preferable in combination with water, it is also possible to use so-called physical blowing agents. These are compounds which are inert towards the feed components and are mostly liquid at room temperature and vaporize under the conditions of the urethane reaction. Preferably, the boiling point of these compounds is below 110° C., in particular below 80° C. Physical blowing agents also include inert gases, which are introduced into the feed components and/or dissolved therein, for example carbon dioxide, nitrogen or noble gases.

The compounds that are liquid at room temperature are mostly selected from the group comprising alkanes and/or cycloalkanes having at least 4 carbon atoms, dialkyl ethers, esters, ketones, acetals, fluoroalkanes having 1 to 8 carbon atoms, and tetraalkyl-silanes having 1 to 3 carbon atoms in the alkyl chain, in particular tetramethylsilane.

Examples which may be mentioned are propane, n-butane, iso- and cyclobutane, n-, iso- and cyclopentane, cyclohexane, dimethyl ether, methyl ethyl ether, methyl butyl ether, methyl formate, acetone, and also fluoroalkanes, which can be degraded in the troposphere and therefore are not harmful to the ozone layer, such as trifluoromethane, difluoromethane, 1,1,1,3,3-pentafluorobutane, 1,1,1,3,3-pentafluoropropane, 1,1,1,2-tetrafluoroethane, difluoroethane and heptafluoropropane. The specified physical blowing agents can be used alone or in any desired combinations.

The catalysts used are in particular compounds which greatly increase the rate of the reaction of the isocyanate groups with the groups that are reactive with isocyanate groups. In particular, organic metal compounds, preferably organic tin compounds, such as tin(II) salts of organic acids, are used.

Furthermore, strongly basic amines can be used as catalysts. Examples thereof are secondary aliphatic amines, imidazoles, am idines, triazines, and alkanolamines.

The catalysts can be used alone or in any desired mixtures with one another, according to requirements.

The auxiliaries and/or additives used are the substances known per se for this purpose, for example surface-active substances, foam stabilizers, cell regulators, fillers, pigments, dyes, flame retardants, hydrolysis inhibitors, antistatics, fungistatic and bacteriostatic agents.

Further details on the starting materials, blowing agents, catalysts and also auxiliaries and/or additives used for carrying out the method according to the invention can be found, for example, in Kunststoffhandbuch [Plastics handbook], volume 7, “Polyurethanes” Carl-Hanser-Verlag Munich, 1st edition, 1966, 2nd edition, 1983 and 3rd edition, 1993.

The advantage of the method according to the invention over the epoxidation/ring-opening or the hydroformylation/hydrogenation consists in the fact that no solvents and no catalysts are required for the ketonization process. Consequently, a comparatively cost-effective access to hydroxy-functionalized fats and fatty acid derivatives is possible. Additionally, there is the advantage that, by virtue of simple adaptation of the reaction conditions such as pressure, temperature and residence time, it is possible to adjust functionalities easily and in a targeted manner, and consequently materials are accessible which offer very broad application possibilities, which also extend beyond polyurethane applications.

Compared with the epoxidation and the ozonolysis, this method offers the advantage of generating oligohydroxy fats which no longer comprise double bonds coupled with freely adjustable degree of hydroxylation and are thus no longer subject to the customary ageing process of fats (oxidation of the DB, “rancidification”). In the case of epoxidation or ozonolysis, this occurs only in the event of complete conversion but this determines the degree of functionalization.

Compared to the hydroformylation, the nitrous oxide oxidation permits the production of material with complementary reactivity since here exclusively secondary hydroxy groups are generated, whereas the hydroformylation produces primary OH groups.

By virtue of the subsequent addition reaction of the alkylene oxides it is possible to optimize the polyols for their particular intended use. For example, for polyols which are intended for use in flexible polyurethane foams, longer chains are added on than in the case of those for use in rigid polyurethane foams.

The invention will be illustrated in more detail by reference to the examples below.

### EXAMPLE 1

### Oxidation of Soya Oil With Nitrous Oxide

260 g of soya oil were charged to a steel autoclave with a capacity of 1.2 L, and the autoclave was closed and rendered inert with nitrogen. 50 bar of nitrous oxide were injected, the stirrer was set at 700 rpm and switched on and then the reaction mixture was heated to 220° C. After a run time of 22 h, the mixture was cooled to room temperature, the stirrer was switched off and the system was slowly decompressed to ambient pressure. After removing the solvent, the yellowish liquid product was analyzed.

Analytical data: bromine number 36 g bromine/100 g, carbonyl number 173 mg KOH/g, ester number 196 mg KOH/g, acid number 1.8 mg KOH/g. Elemental analysis: C=73.6%, H=10.8%, O=15.1%.

### EXAMPLE 2

### Oxidation of Soya Oil With Nitrous Oxide

172 g of soya oil and 172 g of cyclohexane were charged to a steel autoclave with a capacity of 1.2 L, and the autoclave was closed and rendered inert with nitrogen.

20 bar of nitrous oxide were injected, the stirrer was set at 700 rpm and switched on, and then the reaction mixture was heated to 220° C. After a run time of 36 h, the mixture was cooled to room temperature, the stirrer was switched off, and the system was slowly decompressed to ambient pressure. After removing the solvent, the yellowish liquid product was analyzed.

Analytical data: bromine number 57 g bromine/100 g, carbonyl number 64 mg KOH/g, ester number 196 mg KOH/g, acid number 1.8 mg KOH/g. Elemental analysis: C=75.6%, H=11.5%, O=13.4%.

### EXAMPLE 3

### Oxidation of Soya Oil With Nitrous Oxide in the Tubular Reactor

At 290° C. and 100 bar, 130 g/h of a mixture of 50% by weight soya oil and 50% by weight cyclohexane were reacted with 45 g/h of nitrous oxide in a tubular reactor (capacity 210 ml, residence time ca. 50 min). The reaction product was decompressed in a container, the liquid fraction of the reaction product was cooled and the cyclohexane was removed by distillation. The yellowish liquid product was analyzed. Analytical data: bromine number 54 g bromine/100 g, carbonyl number 81 mg KOH/g, ester number 199 mg KOH/g, acid number 2.6 mg KOH/g. Elemental analysis: C=75.0%, H=11.1%, O=13.7%.

The soya oil used in all examples was a commercial product from Aldrich with a bromine number of 80 g bromine/100 g, a carbonyl number of 1 mg KOH/100 g, a saponification number of 192 mg KOH/g and an acid number of <0.1 mg KOH/g. Elemental analysis revealed C=77.6%, H=11.7%, O=11.0%.

### EXAMPLE 4

### Hydrogenation of the Oxidized Soya Oil From Example 2

A solution of 20 g of oxidized soya oil from Example 2 (carbonyl number=64, OH number<5, bromine number=57) in 100 ml of tetrahydrofuran is introduced as initial charge in a 300 ml steel autoclave together with 2 g of a water-moist, 5% ruthenium catalyst on a carbon support. The solution was heated to 120° C., and 120 bar of hydrogen were injected. At these parameters, the mixture was stirred for 12 h. The reaction mixture was then cooled and decompressed. The product was filtered and the solvent is removed by distillation. Analysis of the solid (butter-like) residue revealed an OH number of 64, a carbonyl number <5 and a bromine number of <5.

### EXAMPLE 5

### Hydrogenation of the Oxidized Soya Oil From Example 3

A solution of 20 g of oxidized soya oil (carbonyl number=81, bromine number=54) in 100 ml of tetrahydrofuran was introduced as initial charge in a 300 ml steel autoclave together with 20 g of a water-moist, Al₂O₃-supported ruthenium catalyst (0.5%). The solution was heated to 120° C., and 100 bar of hydrogen were injected. At these parameters, the solution was stirred for 12 h. The reaction mixture was then cooled and decompressed. The reaction product was filtered and then the solvent was removed by distillation. Analysis of the solid (butter-like) residue revealed an OH number of 80, a carbonyl number <5 and a bromine number of <5.

### EXAMPLE 6

### Hydrogenation of the Oxidized Soya Oil From Example 1

A solution of 20 g of oxidized soya oil from Example 1 (carbonyl number=173, OH number<5, bromine number=36) in 100 ml of tetrahydrofuran was introduced as initial charge in a 300 ml steel autoclave together with 2 g of a water-moist, 5% ruthenium catalyst on a carbon support. The solution was heated to 120° C., and 120 bar of hydrogen were injected. At these parameters, the solution was stirred for 12 h. The reaction mixture was then cooled and decompressed. The product was filtered and then the solvent was removed by distillation. Analysis of the solid (butter-like) residue revealed an OH number of 170, a carbonyl number <5 and a bromine number of <5.

The polyol from Example 6 was used in a rigid polyurethane foam formulation. In this connection, it was established that the system was characterized by excellent compatibility with the pentane used as blowing agent.

### EXAMPLE 7

### Alkoxylation of Hydroxy-Soya Oil From Example 6

1523 g of hydroxy oil from Example 6 (OH number=170 mg KOH/g) were introduced as initial charge in a pressurized autoclave and admixed with 11.5 g of a 5.4% strength suspension of a zinc hexacyanocobaltate in Lupranol® 1100. After the reaction mixture had been rendered inert three times with nitrogen, the reaction mixture was freed from the water under reduced pressure at 20 mbar for ca. 30 minutes at 130° C. Then, firstly to activate the catalyst, 150 g of propylene oxide were metered into the reaction mixture over the course of 10 minutes. After the activation, which was evident from a temperature increase in combination with a significant pressure drop, a further 3720 g of propylene oxide were metered into the reaction mixture over the course of 160 minutes. When the metered addition of the monomer was complete and after a constant reactor pressure had been reached, unreacted propylene oxide and other volatile constituents were distilled off in vacuo, and the product was drained off. In this way, 5300 g of the desired product were obtained in the form of a slightly yellowish, viscous liquid with an OH number of 50.6 mg KOH/g and a viscosity of 842 mPas.

The polyol from Example 7 was used in a flexible polyurethane foam formulation. Here, the polyol was used as the only polyol. There were no negative effects at all on the processability of the system or on the mechanical parameters of the flexible foam.

### EXAMPLE 8

### Alkoxylation of Hydroxy-Soya Oil From Example 5

917 g of hydroxy oil from Example 5 (OH number=80 mg KOH/g) were introduced as initial charge in a pressurized autoclave and admixed with 6.42 g of a 5.7% strength suspension of a zinc hexacyanocobaltate in Lupranol® 1100. After the reaction mixture had been rendered inert three times with nitrogen, the reaction mixture was freed from the water under reduced pressure at 20 mbar for ca. 30 minutes at 130° C. Then, firstly to activate the catalyst, 50 g of propylene oxide were metered into the reaction mixture over the course of 10 minutes. After the activation, which was evident from a temperature increase in combination with a significant pressure drop, a further 500 g of propylene oxide were metered into the reaction mixture over the course of 100 minutes. When the metered addition of the monomer was complete and after a constant reactor pressure had been reached, unreacted propylene oxide and other volatile constituents were distilled off in vacuo, and the product was drained off. In this way, 1350 g of the desired product were obtained in the form of a slightly yellowish, viscous liquid with an OH number of 49.8 mg KOH/g and a viscosity of 527 mPas.

The polyol from Example 8 was used in a polyurethane center shoe sole formulation. Here, the polyol was used as the only polyol. The products obtained were characterized moreover by an improved surface nature.

The polyol from Example 8 was also used in a polyurethane sealant formulation. The sealants obtained were characterized by excellent hydrolysis stabilities.

1. A method for producing a polyol, the method comprising:
(a) reacting at least one selected from the group consisting of an unsaturated natural fat, an unsaturated natural fatty acid, and a fatty acid ester with dinitrogen monoxide, to obtain a first intermediate;
(b) reacting the first intermediate with a hydrogenation reagent, to obtain a second intermediate;
(c) reacting the second intermediate with at least one alkylene oxide.

2. The method of claim 1, wherein the unsaturated natural fat is selected from the group consisting of castor oil, grapeseed oil, black caraway oil, pumpkin seed oil, borage seed oil, soya oil, wheat germ oil, rapeseed oil, sunflower oil, peanut oil, apricot kernel oil, pistachio kernel oil, almond oil, olive oil, macadamia nut oil, avocado oil, sea buckthorn oil, sesame oil, hemp oil, hazelnut oil, evening primrose oil, wild rose oil, safflower oil, walnut oil, palm oil, fish oil, coconut oil, tall oil, corn germ oil, and linseed oil.

3. The method of claim 1, wherein the fatty acid is selected from the group consisting of myristoleic acid, palmitoleic acid, oleic acid, vaccenic acid, petroselinic acid, gadoleic acid, erucic acid, nervonic acid, linoleic acid, α-linolenic acid, γ-linolenic acid, stearidonic acid, arachidonic acid, timnodonic acid, clupanodonic acid, and cervonic acid.

4. The method of claim 1, wherein the unsaturated natural fat is selected from the group consisting of soya oil, palm oil, sunflower oil, and rapeseed oil.

5. The method of claim 1, wherein the dinitrogen monoxide is present in a mixture with at least one inert gas.

6. The method claim 1, wherein the hydrogenation reagent is a complex metal hydride.

7. The method of claim 1, wherein the hydrogenation reagent is lithium aluminum hydride, sodium borohydride, or lithium borohydride.

8. The method of claim 1, wherein the hydrogenation reagent is hydrogen.

9. The method of claim 8, wherein the reacting (b) is carried out in the presence of a catalyst.

10. The method of claim 8, wherein the reacting (b) is carried out in the presence of a catalyst comprising a transition metal of groups 6 to 11.

11. The method of claim 8, wherein the reacting (b) is carried out in the presence of a catalyst comprising ruthenium.

12. The method of claim 8, wherein the reacting (b) is carried out in the presence of a catalyst comprising nickel.

13. The method of claim 1, wherein the reacting (c) is carried out in the presence of a catalyst.

14. The method of claim 1, wherein the reacting (c) is carried out in the presence of a multi-metal cyanide catalyst.

15. A polyol obtained by the process of claim 1.

16. (canceled)

17. A method for producing a polyurethane, the method comprising:
reacting at least one polyisocyanate with at least one compound comprising two hydrogen atoms that are reactive with an isocyanate group,
wherein the at least one compound is a polyol of claim 15.

18. The method of claim 1, wherein the fatty acid ester is an ester of a fatty acid selected from the group consisting of myristoleic acid, palmitoleic acid, oleic acid, vaccenic acid, petroselinic acid, gadoleic acid, erucic acid, nervonic acid, linoleic acid, α-linolenic acid, γ-linolenic acid, stearidonic acid, arachidonic acid, timnodonic acid, clupanodonic acid, and cervonic acid.

19. The method of claim 10, wherein the transition metal is selected from the group consisting of copper, molybdenum, palladium, and platinum.

20. The method of claim 1, wherein the alkylene oxide is at least one selected from the group consisting of ethylene oxide and propylene oxide.

21. The method of claim 14, wherein a content of the multi-metal catalyst is from 50-150 ppm, based on a total amount of the polyol.
